(19) United States
(12) Patent Application Publication
(10) Pub. No.: US 2016/0045112 A1
(43) Pub. Date: Feb. 18, 2016

(54) REDUCING INTERFERENCE IN A COMBINED SYSTEM COMPRISING AN MRI SYSTEM AND A NON-MR IMAGING SYSTEM

(71) Applicant: KONINKLIJKE PHILIPS N.V., EINDHOVEN (NL)

(72) Inventors: Bjoern WEISSLER, Aachen (DE); Volkmar SCHULZ, Wuerselen (DE); Pierre Klaus GEBHARDT, Aachen (DE); Peter Michael Jakob DUEPPENBECKER, Aachen (DE); Christoph Werner LERCHE, Herzogenrath (DE)

(21) Appl. No.: 14/436,489

(22) PCT Filed: Oct. 28, 2013
(86) PCT No.: PCT/EP2013/072469

§ 371 Date: Apr. 17, 2015

(30) Foreign Application Priority Data
Oct. 26, 2012 (EP) 12190091.4

(51) Int. Cl. A61B 5/00; G01R 33/00; A61B 6/00; A61B 5/055; A61B 6/03

(52) U.S. Cl. CPC A61B 5/0035; A61B 5/055; A61B 6/037; A61B 6/4417; A61B 6/5205; A61B 6/54; G01R 33/0029; A61B 2560/0204; A61B 2560/0214

(57) ABSTRACT

The present invention relates to a method and a system for reducing interference between a non-MR imaging system (e.g. a PET imaging scanner) and an MR imaging system. The method comprises receiving at least a signal indicative of the MR RF signal detection period, and in response to the received signal, setting the state of at least a portion of the non-MR imaging system to an inactive state during at least a portion of the MR RF signal detection period.

### FIELD OF THE INVENTION

The invention relates to a method and a system for use in combined imaging systems that include Magnetic Resonance (MR) imaging. The invention finds particular application in PET-MR imaging systems, even more particularly in such imaging systems that acquire images substantially simultaneously.

### BACKGROUND OF THE INVENTION

In the field of MR imaging the demand for improved medical diagnosis has led to the development of so-called combined imaging systems. Such imaging systems augment the soft-tissue image contrast benefits of MR with for example the functional imaging capabilities of PET or SPECT. However the design of combined imaging systems is frustrated by interoperability constraints. The few-Tesla magnetic fields and the high RF fields generated within the MR imaging system bore restrict the design freedom in the non-MR imaging system with which it is combined, limiting for example the range of materials that can be used in the non-MR system. Furthermore the proximity between the two imaging systems risks interference from one system degrading the other's performance.

Combined imaging systems may be formed through co-location, in which a non-MR imaging system is placed close to an MR imaging system. During operation, a patient bed is translated between the two imaging systems and images are acquired consecutively. The separation between the imaging systems relaxes the impact of one system on the other but risks patient motion between the consecutive acquisitions degrading image quality. Combined imaging systems may also be fully integrated, in which an MR imaging system is combined with a non-MR imaging system in the same housing offering both simultaneous acquisition and a reduction of image artefacts at the expense of aggravated interoperability issues.

A particular interoperability issue found in combined imaging systems that include an MR imaging system and a non-MR imaging system is that of electrical interference between the non-MR imaging system and the MR imaging system. In this, electrical currents flowing in the circuits of the non-MR imaging system produce electromagnetic radiation which risks being detected by the sensitive RF sense coils in the MR imaging system. The problem is particularly acute in simultaneous-acquisition systems in which a shared imaging region necessarily requires some parts of the non-MR imaging system to be located close to the bore of the MR imaging system where the sensitive RF coils are located. The RF sense coils are typically only sensitive to a particular frequency bandwidth, thus only frequencies within this bandwidth present an issue. However digital signals which may be used in the non-MR imaging system to improve signal integrity or to facilitate signal processing have an inherently broad RF emission spectral bandwidth which may fall within the reception bandwidth of the MR RF receive coil and thereby interfere with the MR imaging system.

Electrical screening is a well-established method of reducing such emissions in combined imaging systems. In this, RF emissions are reduced by surrounding the radiating circuitry with a conductive screen. However the placement of such a screen close to the bore of an MR system risks distorting its magnetic field and degrading the MR image quality. A thicker, more conductive screen reduces the RF emissions at the expense of increased distortion in the MRI images, placing a limit on the effectiveness of electrical screening in this environment. The effectiveness of such a screen is furthermore compromised by the need for openings in the screen to permit for example cooling and data transfer to the electronic circuitry therein.

Another technique for reducing interference between a non-MR imaging system and an MR imaging system is disclosed in US20120089007A1. US20120089007A1 discloses to reduce such interference by using a common timebase between the non-MR imaging system and the MR imaging system.

Document “Interleaved magnetic resonance and ultrasound by electronic synchronisation”, Sebok D A et al, Investigative Radiology, Philadelphia, Pa., US, Vol. 26 no. 4, 1 Apr. 1991, pp 353-357 discloses a technique in which ultrasound gating is made to peacefully co-exist with MRI by gating the ultrasound so that it is disabled during the time of the MR data acquisition.

Patent application US2009/195249A1 by Demeester et al discloses a PET detector ring comprising a radiation detector ring comprising scintillators viewed by photomultiplier tubes, and a magnetic field shielding enclosure which shields the photomultiplier tubes of the radiation detector ring. The PET detector ring may be part of a hybrid imaging system also including a magnetic resonance scanner.

Patent application US2008/169812A1 by ladebeck et al discloses a tomographic measuring system with two tomographic measuring devices, of which a first can interfere with a second in a manner disadvantageous for conducting measurements. In one embodiment the system includes a switching-off mechanism in the first measuring device, and an external connection to the first measuring device for transmitting a switching-off signal to the switching-off mechanism if the second measuring device is conducting a measurement.

Whilst the above-mentioned approaches go some way to reducing interference between a non-MR imaging system and an MR imaging system, the demand for improved quality MR images to further improve patient diagnosis requires this interference to be reduced even further.

### SUMMARY OF THE INVENTION

It is an object of the invention to provide a method and a system for reducing interference between a non-MR imaging system and a nearby MR imaging system.

This object is achieved by the method in which the MR imaging system is defined to have an MR RF signal detection period during which the MR imaging system detects RF signals indicative of the spin of protons within the MR imaging region. Further, at least a portion of the non-MR imaging system is defined to have an active state and an inactive state. The method comprises receiving at least a signal indicative of the MR RF signal detection period; and in response to the received signal, setting the state of at least a portion of the non-MR imaging system to the inactive state during at least a portion of the MR RF signal detection period. In this way, interference between the non-MR imaging system and the MR imaging system is reduced.

In operation, a powerful constant magnetic field in the bore of an MR imaging system aligns the magnetic moments of protons, particularly within water molecules, causing them to spin about an axis parallel to the bore. Pulsed, magnetic fields are generated by RF field coils within the bore and periodically modify the spin characteristics of the protons in order to spatially encode their position. During the RF field's off state the protons return to their aligned positions and gradient fields are applied which encode the spatial positions of protons within the bore. Subsequently the RF receiver coils are switched on for an MR RF signal detection period in order detect the MR signal. Consequently during the MR RF signal detection period, the RF receive coils are particularly sensitive to RF interference. By setting the state of at least a portion of the non-MR imaging system to an inactive state during at least a portion of the MR RF signal detection period the interference between the non-MR imaging system and the MR imaging system is reduced.

The known method of reducing interference between a non-MR imaging system and an MR imaging system disclosed in US20120089007A1 is to synchronise the timing of the two systems using a common timebase. This known method offers some reduction in interference. Such synchronisation methods lock the frequencies of signals in the two systems that would otherwise vary independently and cause interference with variable beat frequencies characteristic of digital noise. The present invention augments the known method by furthermore preventing interference-generating operations in the non-MR imaging system during the MR RF signal detection period when the MR imaging system is particularly sensitive to RF interference. Consequently a further reduction in interference is obtained and the image quality of the MR imaging system is improved. Whilst such an approach in which one imaging system interrupts the operation of another may appear unattractive, risking that such interruptions impact its image quality, in practice the short duty cycle of the MR RF signal detection period means that the non-MR imaging system is only set in the inactive state for short periods thereby having minimal impact upon its performance.

According to one aspect of the invention the MR imaging system has a bore and the inactive state of the non-MR imaging system corresponds to a state in which at least one of the following is switched off: i) the transmission of data from within the bore of the MR imaging system to beyond the bore of the MR imaging system; ii) a clock signal controlling a data processor or sensor within the bore of the MR imaging system; iii) the processing of data within the bore of the MR imaging system; iv) the transfer of data to a memory within the bore of the MR imaging system; v) the generation of timestamps corresponding to the time of detection of gamma photons; vi) the conversion of data from a gamma photon detector in the non-MR imaging system from analogue data to digital data; vii) the transfer of power from beyond the bore of the MR imaging system to a portion of the non-MR imaging system within the bore; viii) the supply of power to at least a portion of the non-MR imaging system. The aforementioned operations may interfere with the MR imaging system and thus their suspension by turning one or more of them off during the MR RF signal detection period reduces such interference.

According to another aspect of the invention the signal indicative of the MR RF signal detection period is or is derived from at least one of the following: i) a tune signal 30 from an MR RF coil in the MR imaging system ii) a gradient field in the MR imaging system iii) a readout gradient field 31 in the MR imaging system iv) a signal from the MR imaging system indicating the receive state of the coils v) a synchronisation signal 32 from the MR imaging system. These signals are typically available within an MR imaging systems and can be advantageously used to determine when to keep elements of the non-MR imaging system in an inactive state.

According to another aspect of the invention, data from at least one of the following sources is buffered during at least a portion of the MR RF signal detection period: i) data indicative of the energy of a received gamma photon ii) data indicative of the time of reception of a received gamma photon iii) control or status data. Preferably the buffering operation takes place locally to the PET gamma photon detector that generates the data, and even more preferably the buffering occurs close to the imaging region of the combined imaging system. In so doing the radiative fields generated by electrical currents associated with data transmission, or those generated by electrical currents associated with the modulation of data onto a carrier frequency in order to facilitate its transmission are inhibited and the interference to the MR imaging system is reduced.

According to another aspect of the invention the state of at least a portion of the non-MR imaging system is set to the active state outside the MR RF signal detection period; thus when the MR imaging system is not detecting RF signals. In so doing the temporary switch-off of interference-generating portions of the PET imaging system is terminated and such portions are returned to their uninterrupted state of operation, thereby allowing for the transmission of data, the transfer of power, the sampling of gamma photon detector data and so on.

According to another aspect of the invention a signal indicative of a preparation phase of the MR imaging system is further received, and in response to this signal the state of at least a portion of the non-MR imaging system is set to the inactive state for at least a portion of a period when the MR imaging system is in the preparation phase. The preparation phase of the MR imaging system is also a period during which the MR imaging system is particularly sensitive to RF emissions. Thus by setting the state of at least a portion of the non-MR imaging system into an inactive state during this period a further reduction in interference with the MR imaging system is achieved.

In accordance with other aspects of the invention, a system and a computer-readable medium are provided in order to implement the method of the invention.

### BRIEF DESCRIPTION OF THE FIGURES

FIG. 1 illustrates a combined PET-MR imaging system in accordance with some aspects of the invention

FIG. 2 illustrates an example implementation of the non-MR Activity Control Unit configured to set interference-generating portions of the non-MR imaging system into an inactive state.

FIG. 3 illustrates an example modulation scheme used in accordance with some aspects of the invention.

FIG. 4 illustrates an example use of the demodulated gate signal 35 and the PET reference clock signal 38 within PET detector module 40a to set an interference-generating portion of the PET detector electronics into an inactive state.

FIG. 5 illustrates an example implementation for storing data suspended from transmission during the inactive state

### DETAILED DESCRIPTION OF THE INVENTION

In order to provide a method and a system for reducing interference between a non-MR imaging system and a nearby MR imaging system, the invention is described with reference to an exemplary combined PET-MR imaging system having substantially simultaneous data acquisition. It should however be appreciated that the invention also has application to combined imaging system in which an MR imaging system is co-located with a non-MR imaging system, and also to imaging systems that combine MR imaging with other imaging modalities. Such combinations include but are not limited to SPECT-MR, optical-MR imaging systems such as bioluminescence-MR, and ultrasound-MR.

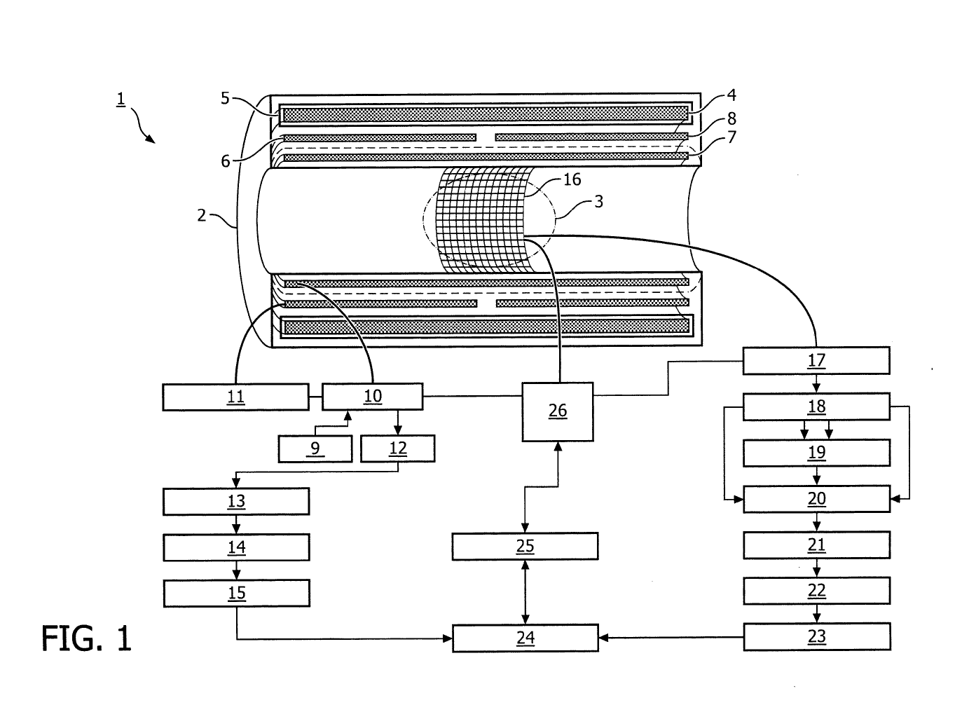

FIG. 1 illustrates a combined PET-MR imaging system in accordance with some aspects of the invention. With reference to FIG. 1, combined PET-MR imaging system 1 comprises a common scanner housing 2 defining an imaging region 3 within which a subject to be imaged such as a human or animal may be located. A main magnet 4 surrounded by cryoshielding 5 generates a main magnetic field in imaging region 3. Magnetic gradient field coils 6 are arranged on or in the housing 2 to generate additional magnetic fields to superimpose upon the main magnetic field in imaging region 3. The magnetic gradient field coils 6 typically include coils for producing three orthogonal magnetic field gradients. In some embodiments a whole-body RF coil 7 with an RF screen 8 is arranged in or on the housing 2 in order to inject RF excitation pulses into the imaging region 3. In other embodiments local coils not shown are used to inject RF pulses local to the subject being imaged.

During MRI image acquisition an RF transmitter 9 is coupled to the whole body coil 7 via RF switching circuitry 10 or coupled to one or more local coils not shown to generate magnetic resonances in a region of the imaging region 3. A gradients controller 11 controls the signals to magnetic field gradient coils 6 in order to spatially encode the magnetic resonances. In one example, a one-dimensional magnetic field gradient applied during radio frequency excitation produces slice-sensitive excitation; magnetic field gradients applied between excitation and readout of magnetic resonances provide phase encoding, and magnetic field gradients applied during readout of magnetic of magnetic resonances provide frequency encoding. The MRI pulse sequences can be configured to produce Cartesian, radial or other spatial encodings.

After the RF excitation the RF switching circuitry 10 operatively disconnects the RF transmitter 9 and connects an RF receiver 12 to the whole body RF coil 7 to acquire spatially-encoded magnetic resonances from within the imaging region 3. Alternatively the RF receiver 12 is connected to one or more of the local coils not shown. The acquired magnetic resonances are stored in MRI data buffer 13 and are reconstructed by an MRI reconstruction processor 14, resulting in a reconstructed MRI image that is stored in MRI images memory 15. The MRI reconstruction processor 14 uses algorithms such as Fast Fourier Transform (FFT) reconstruction algorithms when Cartesian encoding is used.

The combined PET-MR imaging system 1 further includes PET imaging functionality via a plurality of gamma photon detectors 16 disposed radially around the imaging region 3 in order to receive gamma photons emitted therein. Whilst in FIG. 1 the radiation detectors are illustrated within the inner lining of the MR imaging system, other configurations are also contemplated including the location of the radiation detectors within a gap in cylindrical main magnet 4. In another contemplated configuration the PET gamma photon detectors form part of a module that is removably inserted into the bore of the MR imaging system for use in pre-clinical PET-MR imaging.

In PET imaging a radiotracer is administered to a subject such as a patient or an animal prior to its positioning in the imaging region 3. The radiotracer is preferentially absorbed by regions in the subject and its distribution is imaged following an uptake period. The radiotracer undergoes radioactive decay which results in the production of positrons. Each decay event produces one positron which travels up to a few mm in human tissue where it subsequently interacts with an electron in an annihilation event that produces two oppositely-directed gamma photons. The two gamma photons each have an energy of 511 keV and are subsequently detected by the plurality of gamma photon detectors 16 disposed radially around the imaging region 3, each of which produce an electrical signal when struck by a gamma photon. In the embodiment shown in FIG. 1 the electrical signals indicative of received gamma photons are transferred to a PET events buffer located beyond the imaging region. In alternative implementations the events buffer is local to the gamma photon detectors 16 and therefore more proximal to the imaging region 3. The data in PET events buffer 17 is preferably in list mode format and includes at least information indicative of the time of reception of a plurality of gamma photons. The time information may be an absolute time or alternatively each event may be identified as a member of a pair of gamma photons that have been detected substantially simultaneously. The data may further include information indicative of the energy of the received gamma photons. Coincidence determination unit 18 in operative communication with PET events buffer 17 sorts the data into pairs of coincident events that are received substantially simultaneously. Two gamma photons are identified as coincident if their timestamps occur within a narrow time interval of each other; typically if they are detected within +/−3 ns. The positions of the two detectors receiving the coincident gamma photons define a line in space along which the annihilation event occurred, the line being termed a line of response (LOR). The pairs of coincident events from coincidence determination unit 18 are transferred to LOR processor 19 which identifies the spatial LOR along which the event occurred. In time-of-flight (TOF) PET the small time difference between the two detected events is further used to localise the position along the LOR at which the annihilation event occurred, and thus more accurately locate the spatial position of the radiotracer causing the decay event. If the absolute time of the received event is generated, optional TOF processor 20 uses the time difference between the events in each pair to more accurately locate the spatial position of the radiotracer causing the decay event. The resulting data is a PET projection data set 21 which is reconstructed by PET reconstruction processor 22 into a PET image illustrative of the distribution of the radiotracer within the imaging region using techniques such as filtered back projection and iterative reconstruction. The resulting PET image is stored in PET images memory 23. Subsequently the data from the MR and the PET imaging modalities may be processed by post reconstruction image processor 24 for example to align the images, to segment the images into different anatomical compartments, and to determine the radiotracer uptake within the compartments and the like. User interface 25 allows user interaction with the scanning process and with the post image reconstruction processor 24 for example to permit the user to align and manipulate images, to start and stop scans, to set scan parameters such as the scan time, the nature of the RF gradient fields used in the MR imaging process, and to identify the extent of the imaging region to be scanned.

In the embodiment shown in FIG. 1, at least the gamma photon detectors 16 of the PET imaging system are typically located close to the imaging region from which they receive gamma photons. Control circuitry and local data processing circuitry not shown but associated with gamma photon detectors 12 may also be located close to the gamma photon detectors in order to maintain signal integrity and improve system compactness. Events buffer 17 may also be located close to the imaging region. However the operation of electronic circuitry close to the bore of the MR imaging system risks that electromagnetic emissions caused by such electronic circuitry is picked up by the sensitive whole body RF coil 7, or alternatively by the one or more of the local coils not shown in FIG. 1, and subsequently detected by RF receiver 12 where the interference is interpreted as a real signal. Such interference can create spurious image artefacts, thereby degrading the resulting MR image quality. It is particularly the interference within the detection bandwidth of RF receiver 12 that causes a problem; however the inherently broadband electrical switching noise generated by the fast transitions of digital electronic circuits used in the gamma photon detection circuitry risks causing such interference. Electrical shielding of such circuitry offers some reduction in this interference but the materials restrictions of the high magnetic field MRI environment limit the efficacy of such approaches, resulting in a tradeoff between the sensitivity of the MR imaging system and the degradation to MR image quality caused by the PET imaging system.

In order to further reduce interference between the non-MR imaging system and the MR imaging system, in a first embodiment of the invention a non-MR imaging system activity control unit 26 which is operatively connected to portions of the non-MR imaging system is configured to set to an inactive state, thus to switch off, portions of the non-MR imaging system electronic circuitry during at least a portion of the MR RF signal detection period during which the MR imaging system detects RF signals indicative of the spin of protons within imaging region 3.

In order to determine when to set such interference-generating portions of the PET imaging system into an inactive, reduced-interference state, as illustrated in FIG. 1, the non-MR imaging system activity control unit 26 receives signals from the MR imaging system indicative of the MR RF signal detection period. These signals may include a signal indicative of or derived from one or more of the following: i) a tune signal 30 from an MRI RF coil in the MR imaging system ii) a gradient field in the MR imaging system iii) a readout gradient field 31 in the MR imaging system iv) a signal from the MR imaging system indicating the receive state of the coils v) a synchronisation signal 32 from the MR imaging system. A tune signal 30 from an MRI RF coil is a signal that is used to adjust the resonant frequency of an MRI RF receive coil in order to receive the MRI signal. The remainder of the time the coil is detuned to prevent it from absorbing RF radiation during the RF excitation phase. In one example implementation disclosed in WO2008/078270 a tune signal is a DC signal that is applied to a p-i-n diode to operatively connect electronic components to adjust the resonant frequency of an RF receive coil. Thus a tune signal from the RF receive coil is indicative of the MR RF signal detection period and thus when interference-generating portions of the PET imaging system are desirably maintained in an inactive state. Alternatively a signal indicative of the period when the interference-generating PET electronics should be maintained in an inactive state may be generated by detecting the gradient fields, such as for example the readout gradient field. Such a signal may be derived by for example sensing these fields using a conductive coil located close to the MR bore. When the RF radiation from the readout gradient field exceeds a predetermined threshold the RF coil is in the MR RF signal detection period and the interference-generating PET electronics is desirably maintained in an inactive state. Alternatively a signal controlling the readout gradient in the MR imaging system may be used directly. Alternatively a signal from the MR imaging system indicating the receive state of the coils may be used. Such a signal may be found for example in the MR imaging system control electronics connected to an MR coil where such a signal is used to select a particular mode of coil operation. Alternatively a signal indicative of the period when the interference-generating PET electronics should be maintained in an inactive state may be derived from a synchronisation signal from the MR system. An example of a suitable signal is an MRI sequence signal.

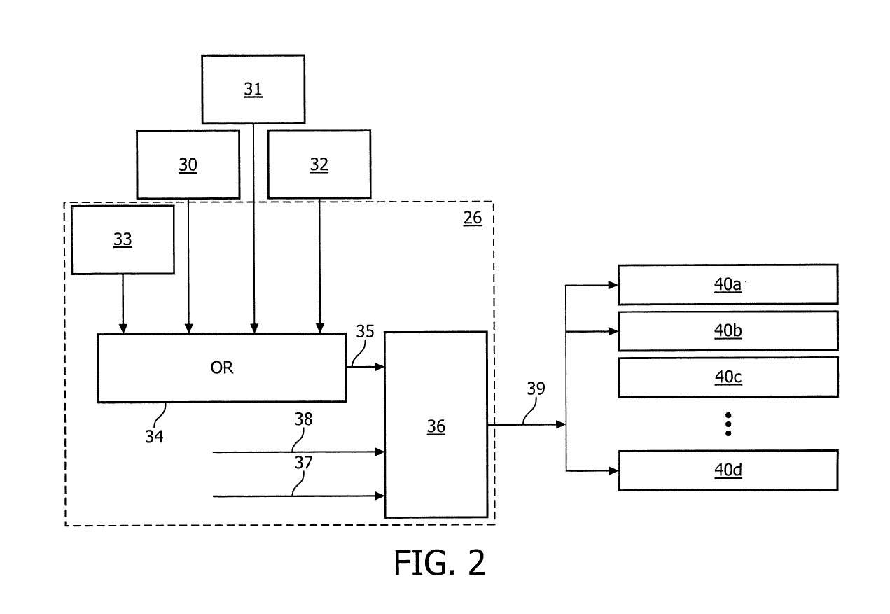

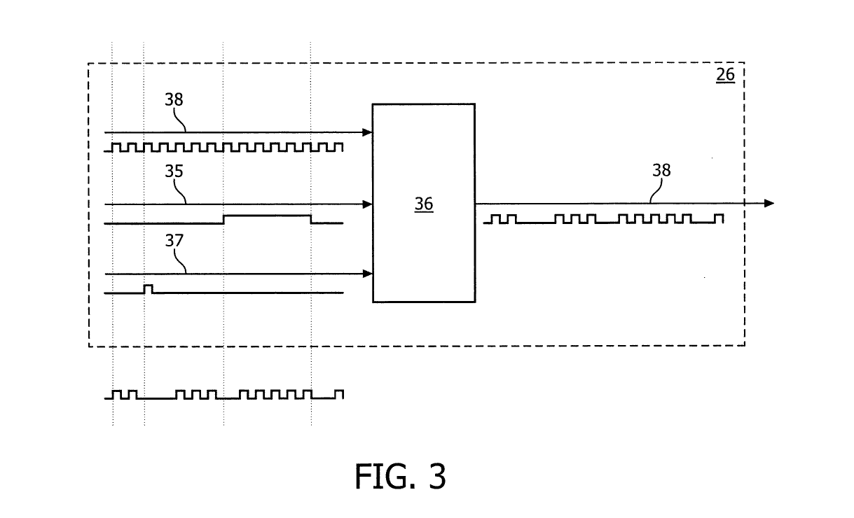

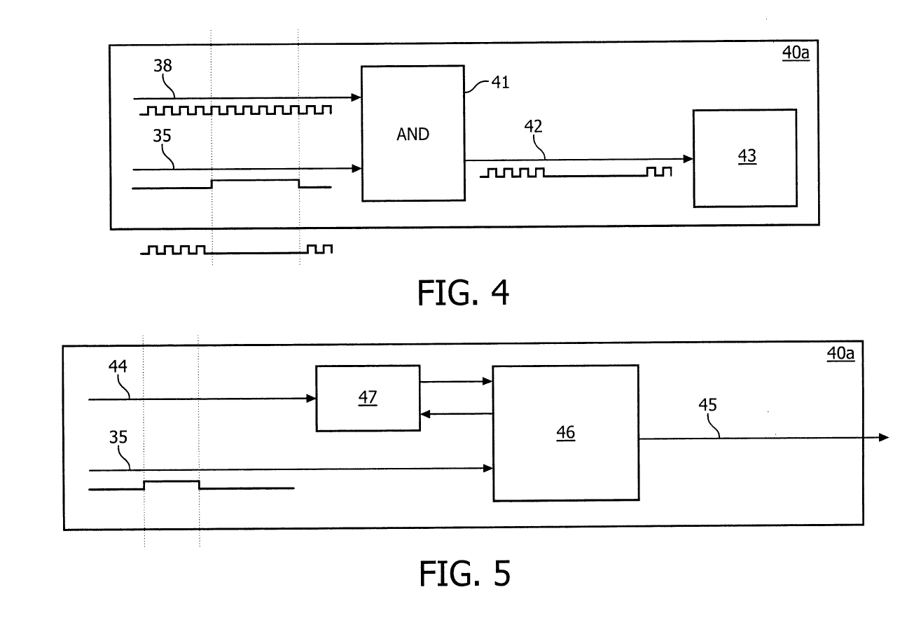

FIG. 2 illustrates an example implementation of the non-MR Activity Control Unit configured to set interference-generating portions of the non-MR imaging system into an inactive state. In FIG. 2, non-MR activity control unit 26 is configured to receive a tune signal 30 from the MRI RF coil, a readout gradient field signal 31, and an external synchronisation signal 32 from the MR imaging system. In other implementations contemplated one or more of these signals may be present. In other implementations contemplated the non-MR activity control unit 26 may be configured to receive a signal indicative of a gradient field in the MR imaging system, or a signal indicative of the receive state of the MR imaging system coils, or any combination of these signals. In FIG. 2, a logical OR of three of these signals in combination with a duty cycle generator 33 generated by OR logic 34 generates gate signal 35 for controlling a modulator 36. Duty cycle generator 33 can be used to inhibit data collection from the PET detector modules when the gamma photon flux is too high for the PET coincidence determination unit to accurately determine coincidence. Modulator 36 modulates gate signal 35, PET synchronisation signal 37 and PET reference clock 38 to generate a composite signal 39 for controlling PET detector modules 40a, 40b, 40c, 40d. FIG. 3 illustrates an example modulation scheme used in accordance with some aspects of the invention. Composite signal 39 may be communicated to the PET detector modules as an electrical signal, or alternatively by means of an optical fiber in order to further reduce interference. PET detector modules 40a, 40b, 40c, 40d subsequently demodulate composite signal 39 to recover the original signals. FIG. 4 illustrates an example use of the demodulated gate signal 35 and the PET reference clock signal 38 within PET detector module 40a to set an interference-generating portion of the PET detector electronics into an inactive state. In FIG. 4, AND logic 41 is used to generate gated sensor clock signal 42 which by virtue of the missing clock cycles temporarily switches off the operation of interference-generating portion 43 of the PET system. In another implementation the gate signal may be used directly to switch off power to an interference-generating portion of the non-MR electronic circuitry or to temporarily set such a portion into a standby state.

In the above examples, portions of the interference-generating PET electronics are maintained in an inactive state by the non-MR imaging system activity control unit for at least a portion of the MR RF signal detection period. By contrast, outside the MR RF signal detection period, the non-MR imaging system activity control unit may be configured to set portions of the interference-generating PET electronics into an active state.

According to one embodiment of the invention the MR imaging system has a bore, and at least one of the following interference-generating portions of the PET imaging system electronics is set into an inactive state, thus switched off, for at least a portion of the MR RF signal detection period: i) the transmission of data from within the bore of the MR imaging system to beyond the bore of the MR imaging system; ii) a clock signal controlling a data processor or sensor within the bore of the MR imaging system; iii) the processing of data within the bore of the MR imaging system; iv) the transfer of data to a memory within the bore of the MR imaging system; v) the generation of timestamps corresponding to the time of detection of gamma photons; vi) the conversion of data from a gamma photon detector in the non-MR imaging system from analogue data to digital data; vii) the transfer of power from beyond the bore of the MR imaging system to a portion of the non-MR imaging system within the bore; viii) the supply of power to at least a portion of the non-MR imaging system. Consequently the interference to the MR imaging system is reduced. The transmission of data from within the bore of the MR imaging system to beyond the bore of the MR imaging system is typically carried out via optical fibers or alternatively via electrical conductors. In both cases the electrical signals required to modulate the signals onto the optical or conductive medium involve electrical currents that risk generating interference. Furthermore the transmission frequencies of such data signals are also typically close to the reception bandwidth of the MR RF receive coils and thus present an elevated risk of causing interference. Therefore a reduction in interference with the MR imaging system may be achieved by switching off transmission of data from within the bore of the MR imaging system to beyond the bore of the MR imaging system. The PET electronics typically employs at least some processing close to the gamma photon detectors and thus close to the bore of the combined PET-MR imaging system. Consequently by switching off a clock signal controlling a data processor or sensor within the bore of the MR imaging system, a substantial reduction in interference may be achieved. Likewise by switching-off the processing of data within the bore of the MR imaging system a reduction in switching transients and therefore interference may be achieved. Other data processing operations including the transfer of data to a memory within the bore of the MR imaging system, the generation of timestamps corresponding to the time of detection of gamma photons, and the conversion of data from a gamma photon detector within the non-MR imaging system from analogue data to digital data also risk generating such interference and are desirably temporarily inhibited by switching off these operations. Switching off any of these operations will therefore reduce interference with the MR imaging system. Furthermore the power supplies used to power the electronic circuitry in the PET electronics may generate interference, particularly in the case of switched-mode power supplies. Therefore by switching-off the transfer of power from beyond the bore of the MR imaging system to a portion of the non-MR imaging system within the bore, a further reduction in the amount of interference with the MR imaging system may be achieved. An alternative method of reducing interference is to switch off the power to a portion of the non-MR imaging system. This may involve turning their power off completely, or setting portions of the non-MR imaging electronics into a low power standby state which has the further benefit of permitting a rapid startup afterwards.

Optionally at least one data buffer is included in the first embodiment illustrated in FIG. 1 wherein the data buffer is configured to buffer data from at least one of the following sources during at least a portion of the MR RF signal detection period: i) data indicative of the energy of a received gamma photon ii) data indicative of the time of reception of a received gamma photon iii) control or status data. The buffer may be implemented for example as a DDR2-memory and advantageously prevents data loss during the suspension of the activity of interference-generating portions of the PET imaging system. Thus when for example the transfer of data from within the bore of the MR imaging system to beyond the bore is temporarily inhibited, for example by storing it to a memory, it is available for transmission during the subsequent phase outside the MR RF signal detection period. FIG. 5 illustrates an example implementation for storing data suspended from transmission during the inactive state. In FIG. 5 sensor data 44 is transmitted from the gamma photon detector to beyond the bore of the MR imaging system by sensor data output 45 via communication interface 46. During the period when portions of the non-MR imaging system in PET detector module 40a are desirably set to an inactive state, demodulated gate signal 35 is high. During this period, communication interface 46 instructs memory 47 to retain data rather than transmitting it to sensor data output 45, thereby buffering the data. When the gate returns to a low state the buffered data is transmitted thereby preventing data loss during the MR RF signal detection period.

According to a second embodiment of the invention, the same interference-generating portions of the non-MR imaging system are furthermore set into an inactive state during the MRI imaging system's preparation phase. Consequently a reduction in interference with the MR imaging system may be achieved. The preparation phase is the time during which the MR system performs various checks and gathers data relating to the next scan. It includes for example checking that the correct MR coil is attached and that all the channels are working; checking for signal correction levels; ensuring that the receiver coil is tuned to receive at the correct frequency and gather data for phase correction. During this period the MRI imaging system is also particularly sensitive to interference from portions of the nearby PET imaging system. The preparation phase may be detected by receiving a signal generated from the select lines of the MRI coil. The select lines control the coil's mode. The signal may be generated using a logical combination of the select lines such that it indicates a mode during the preparation phase when the MR system is sensitive to interference. In response to this signal, portions of the PET imaging system circuitry may be set into an inactive state.

According to a third embodiment of the invention a computer-readable medium having instructions to perform the method of the invention is claimed.

To summarise, a method and a system for use in a combined imaging system comprising an MR imaging system and a non-MR imaging system is provided for reducing interference between the non-MR imaging system and the MR imaging system. The method comprises receiving at least a signal indicative of the MR RF signal detection period, and setting the state of at least a portion of the non-MR imaging system to an inactive state during at least a portion of the MR RF signal detection period.

While the invention has been illustrated and described in detail in the drawings and foregoing description, such illustrations and description are to be considered illustrative or exemplary and not restrictive; the invention is not limited to the disclosed embodiments and can be used in various types of imaging systems.

1. A method for use in a combined imaging system comprising an MR imaging system with a bore and a PET imaging system;
the MR imaging system having an MR RF signal detection period during which the MR imaging system detects RF signals indicative of the spin of protons within the MR imaging region; and
at least a portion of the PET imaging system having an active state and an inactive state;
the method comprising the steps of:
receiving a signal indicative of the MR RF signal detection period; and either;
for the combined imaging system wherein the PET imaging system is fully integrated with the MR imaging system and response to the received signal, setting the state of at least a portion of the PET imaging system to the inactive state during at least a portion of the MR RF signal detection period by switching at least one of the following off:
i) the transmission of PET imaging data from within the bore of the MR imaging system to beyond the bore of the MR imaging system;
ii) a clock signal controlling a PET imaging data processor within the bore of the MR imaging system;
iii) the processing of PET imaging data within the bore of the MR imaging system;
iv) the transfer of PET imaging data to a memory within the bore of the MR imaging system;
v) the transfer of power from beyond the bore of the MR imaging system to a portion of the PET imaging system within the bore;
or, for the combined imaging system wherein the PET imaging system is located close to the MR imaging system and in response to the received signal, setting the state of at least a portion of the PET imaging system to the inactive state during at least a portion of the MR RF signal detection by switching least one of the following of:
vi) the generation of timestamps corresponding to the time of detection of gamma photons;
vii) the conversion of data from a gamma photon detector in the PET imaging system from analogue data to digital data;
viii) the supply of power to at least a portion of the PET imaging system;
wherein the signal indicative of the MR RF signal detection period is either:
i) generated from a readout gradient field in the MR imaging system by sensing its field using a conductive coil located close to the MR bore; or
ii) derived from a detune signal from an MRI RF coil in the MR imaging system.

2. The method according to claim 1 further comprising the buffering of at least one of the following during at least a portion of the MR RF signal detection period:
i) data indicative of the energy of a received gamma photon;
ii) data indicative of the time of reception of a received gamma photon.

3. The method according to claim 1 further comprising the steps of:
receiving a signal indicative of a preparation phase of the MR imaging system
during which the MR imaging system performs at least one of the following operations i) checking that the correct MR coil is attached ii) checking that all channels are working iii) checking that the receiver coil is tuned to receive at the correct frequency; and
in response to the received signal indicative of the preparation phase, setting the state of at least a portion of the PET imaging system to the inactive state for at least a portion of a period when the MR imaging system is in the preparation phase.

4. (canceled)

5. A combined imaging system comprising an MR imaging system with a bore and a PET imaging system; and
a PET imaging system activity control unit in operative communication with the PET imaging system and configured to receive from the MR imaging system at least one MR activity signal indicative of the MR RF signal detection period during which the MR imaging system detects RF signals indicative of the spin of protons within the MR imaging region;
the PET imaging system activity control unit having arithmetic means for comparing the at least one MR activity signal with a threshold activity level; and
at least a portion of the PET imaging system having an active state and an inactive state;
the PET imaging system activity control unit being configured to set the state of at least a portion of the PET imaging system to the inactive state for at least a portion of a period when the at least one activity signal exceeds the threshold activity level by either;
for the combined imaging system wherein the PET imaging system is fully integrated with the MR imaging system, switching at least one of the following of:
i) the transmission of PET imaging data from within the bore of the MR imaging system to beyond the bore of the MR imaging system;
ii) a clock signal controlling a PET imaging data processor within the bore of the MR imaging system;
iii) the processing of PET imaging data within the bore of the MR imaging system;
iv) the transfer of PET imaging data to a memory within the bore of the MR imaging system;
v) the transfer of power from beyond the bore of the MR imaging system to a portion of the PET imaging system within the bore; or
for the combined MR imaging system wherein the PET imaging system is located close to the MR imaging system, switching at least one of the following of:
vi) the generation of timestamps corresponding to the time of detection of gamma photons;
vii) the conversion of data from a gamma photon detector in the PET imaging system from analogue data to digital data;
viii) the supply of power to at least a portion of the PET imaging system; wherein the at least one MR activity signal is either:
i) generated from a gradient field or a readout gradient field in the MR imaging system by sensing its field using a conductive coil located close to the MR bore; or
ii) derived from a detune signal from an MRI RF coil in the MR imaging system.

6. The imaging system according to claim 5 further comprising at least one data buffer configured to buffer data from at least one of the following sources during at least a portion of the MR RF signal detection period: i) data indicative of the energy of a received gamma photon ii) data indicative of the time of reception of a received gamma photon.

7. The imaging system according to claim 5 wherein the at least one MR activity signal further includes a signal indicative of the MR imaging system preparation phase during which the MR imaging system performs at least one of the following operations i) checking that the correct MR coil is attached ii) checking that all channels are working iii) checking that the receiver coil is tuned to receive at the correct frequency; and
wherein in response to the received signal indicative of the preparation phase the PET imaging system activity control unit further configured to set the state of at least a portion of the PET imaging system to the inactive state for at least a portion of a period when the signal indicative of the MR imaging system preparation phase exceeds the threshold activity level.

8. (canceled)

9. A computer-readable medium having instructions to perform the method of claim 1.
